(19) United States
(12) Patent Application Publication
(10) Pub. No.: US 2024/0172614 A1
(43) Pub. Date: May 30, 2024

(54) BREEDING METHOD FOR RAPIDLY STABILIZING COTTON DISTANT HYBRIDIZATION PROGENY

(71) Applicant: Nanyang Academy of Agricultural Sciences, Nanyang (CN)

(72) Inventors: Zongpeng Cao, Nanyang (CN); Qinghua Wang, Nanyang (CN); Lixuan Yang, Nanyang (CN); Peng Dong, Nanyang (CN); Min Li, Nanyang (CN); Yuanchun Qu, Nanyang (CN); Zhen Xiang, Nanyang (CN); Jianding Shen, Nanyang (CN); Xiaofeng Xu, Nanyang (CN); Honglei Quan, Nanyang (CN); Jianbin Shi, Nanyang (CN); Feng Liu, Nanyang (CN)

(21) Appl. No.: 18/401,431

(22) Filed: Dec. 30, 2023

(30) Foreign Application Priority Data
Apr. 13, 2023 (CN) 202310392959.3

(51) Int. Cl. A01H 1/02

(52) U.S. Cl. CPC A01H 1/021; A01H 1/026

(57) ABSTRACT

A breeding method for rapidly stabilizing cotton distant hybridization progeny is disclosed. It belongs to the technical field of plant breeding. The disclosure is based on VSG (G. barbadense semi-compatible material) as female parent, general G. hirsutum materials as male parent of the cross to get F1 generation of G. barbadense-G. hirsutum hybrid materials, and treats the F1 generation of young bolls with 0.03-0.05% of colchicine aqueous solution to induce F2 generation to separate the stable intermediate materials or new varieties with excellent genes of both G. barbadense and G. hirsutum, so as to produce new cotton germplasm resources. The main innovation of the present disclosure lies in that it changes the previous treatment position and method of colchicine on cotton plant, and the disclosure is not limited by haploid and directly treats the cotton bolls on F1 cotton plants.

### TECHNICAL FIELD

The present disclosure relates to the technical field of plant breeding, and more specifically, to a breeding method for rapidly stabilizing cotton distant hybridization progeny.

### BACKGROUND ART

China is the largest producer and consumer of cotton in the world, with an annual demand of about 6 million tons of raw cotton, and its related industries generate more than $300 billion in annual exports. Therefore, stabilizing cotton production plays a pivotal role in promoting Chinese economic development and maintaining national strategic security. In the past decade, with the rapid economic development of China and rising labor costs, coupled with the rapid increase in the level of mechanization of food production, the comparative efficiency of cotton planting is getting lower and lower, the cotton area of the traditional cotton planting areas in the mainland is declining rapidly, and the past cotton fields gradually replaced by corn, peanuts and other crops. Since the 12th Five-Year Plan, the state has made a large-scale adjustment of China's cotton industry, reducing the cotton area in the Yangtze River and Yellow River basins and expanding the cotton planting area in the in G. hirsutum areas of Xinjiang. After ten years of development, Xinjiang has undertaken about 90% of China's cotton production with its advantage of highly intensive management, which has made great contributions to ensuring the strategic safety of China's cotton and resisting the impact of foreign cotton dumping. However, the fiber length of cotton varieties used in Xinjiang production is mostly concentrated between 28 mm and 29 mm, the phenomenon of homogenization is very serious, with the lack of long-staple G. hirsutum varieties longer than 31 mm, and the domestic market gap of more than 2 million tons every year. At present, China's cotton textile enterprises in urgent need of high-quality long-staple cotton raw material sources mainly rely on Australia, the United States and other countries. Because of the single import route, it brings great risks to China's cotton spinning industry. Therefore, there is a bottleneck problem of unreasonable variety structure in cotton production, and breakthroughs need to be made in breeding research. Among them, distant hybridization between G. barbadense and G. hirsutum will be the only way to solve the problem of cotton quality.

Distant hybridization breeding is one of the main technical means of crop breeding, which has achieved significant results in breeding of wheat, rice, corn, peanuts and other major crops, and many varieties bred have been popularized on a large scale because they contain some outstanding advantages, and great economic benefits have been obtained. In nature, G. hirsutum, G. barbadense, G. arboreum, and G. herbaceum are four closely related cultivated species in the cotton genus, among which G. herbaceum and G. arboreum are heterozygous diploid, G. hirsutum and G. barbadense are two heterozygous tetraploid species in the cotton genus. G. hirsutum has become the main variety of cotton production worldwide due to its short growth period, high yield, high lint percentage, and easy management. However, it also has disadvantages such as medium length of fibers, poor strength, high MC value, and can only spin low-end yarns with less than 60 counts. On the contrary, G. barbadense has become a high-quality special cotton variety because of its advantages such as “long fiber, high strength, good disease resistance, moderate MC value, suitable for spinning more than 60 high-end yarns”. However, compared with G. hirsutum varieties, there are shortcomings such as “narrow area of suitability, low yield, small boll, low lint percentage, not suitable for management”. Therefore, the production is only as a special use of matching varieties planted, accounting for only about 10% of the total cotton area. For many years, breeding institutions at all levels have been conducting hybridization breeding between G. barbadense and G. hirsutum, attempting to combine the advantages of both through distant hybridization and breed new varieties of G. hirsutum that possess both excellent traits of G. hirsutum and high-quality disease resistance characteristics of G. barbadense. However, there have been no successful reports. The reason is that the two belong to the same genus but not the same species of closely related plants, and their genetic relationship is too distant. After hybridization, the offspring of the two encounter the problem of “crazy separation, difficult stability”. The results of directed selection often return to the two parents themselves, and it is rare to successfully select intermediate types with stable traits.

To this end, breeders tried to artificially induce G. barbadense-G. hirsutum hybrid F1 generation haploid plants, and then treat these haploid plants with 0.03-0.05% colchicine aqueous solution, so as to double the chromosomes to obtain homozygous G. barbadense-G. hirsutum hybrid “homozygous lines”. The treatment methods include maceration, stem tip injection, agar method, drop method, etc. However, these methods have been successful in other crops, but there has been no breakthrough in cotton breeding. There are two main problems: First, the emergence of cotton haploids in nature only exists in individual varieties of G. barbadense, and the chances are as low as one in 10,000 or less, the F1 generation of haploids with the target traits of G. barbadense-G. hirsutum hybridization is even less. Second, haploids are generally induced by pollen culture, but in cotton has not been induced successfully, there are no cases of double success in occasional haploid plants.

In the early 1960s, American scholars E. L. Turcottet and C. V. Feaster found cotton semi-compatible reproduction phenomenon between fertilization and fusionless reproduction, and transferred the double recessive genes with V7V7 bud yellow trait to the pima cotton (G. barbadense type), so that it not only had the traits of semi-compatible, but also retained the traits of disease resistance and quality of G. barbadense. In the early 1980s, Nanyang Institute of Agricultural Sciences in Henan Province introduced G. barbadense semi-compatible material (VSg: G. barbadense, 2n=52) with V7V7 bud yellow gene into China for the first time, and made a preliminary study and analysis on its growth characteristics and preservation methods.

Crossing cotton semi-compatible material VSG with G. hirsutum breeding material, due to the semi-compatible reproductive characteristics of VSG, more than 90% of normal G. barbadense-G. hirsutum hybrids with about 10% haploids (including three types of haploids of male parent type, female parent type and parental chimeric type) will appear in the hybrid generation. The male parent type haploids appearing in the F1 generation of hybridization are the most useful for breeding work, and by doubling the chromosomes, homozygous diploids of the paternal type can be obtained. However, haploid plants produced using semi-compatible materials have a low chance of doubling in nature and need to be artificially doubled to obtain more DH lines for breeding purposes. The method of artificial doubling by colchicine is usually used. In breeding practice, chromosome doubling can be divided into three cases: (1) Seedling bud immersion method: the tops of haploid seedlings were immersed in 0.05% colchicine solution for 6 hours at 23˜26° C., and the residual colchicine solution was rinsed off with warm water after treatment. (2) Adult bud immersion method: under 28˜30° C., the growth points on the young branches on the haploid plants were immersed in 0.03˜0.05% colchicine plus 5% dimethylphenol solution for 12˜24 h. (3) Injection method: 2 ml of the treatment solution was injected into the bases of the lateral buds by using a microsyringe.

Guo Baode and other researchers at the Institute of Crop Genetics, Shanxi Academy of Agricultural Sciences, took the cotton semi-compatible reproductive material VSG as the female parent and the distant hybrid bud-resistant low-generation material as the male parent for crossbreeding, and selected the paternal-type haplotype haploid appearing in the F1 for chromosome doubling, and selected and bred a new bud-resistant cotton variety, Jin cotton No. 51, with 30.14%, 67.84% and 41.22% reduction in seedling aphid, recovery and volvulus aphid stages aphid index compared to the control, respectively, and the resistance levels all reached grade I. Ji Lixia and other technical personnel used semi-compatible breeding methods to quickly stabilize the distant hybridization method, selected and bred five new distant hybrid germplasm lines of long staple cotton with early maturity, large boll, high yield, high quality fiber and excellent comprehensive characters of G. hirsutum, with 2.5% of the fiber spanning the length of 35.3˜ 37.7 mm, Micronaire value of 3.9˜4.6, yield index than the G. barbadense parent has a substantial improvement, and three germplasm lines yield exceeded the G. hirsutum control variety Jin cotton No. 37. These results have been achieved using two traditional methods of doubling by colchicine, seedling bud immersion method and adult bud immersion method.

In summary, how to provide a breeding method for rapidly stabilizing cotton distant hybridization progeny is a problem that needs to be solved urgently by the technicians in this field.

### SUMMARY

In view of this, the present disclosure provides a breeding method for rapidly stabilizing cotton distant hybridization progeny. The main technical problem solved by the present disclosure is to overcome the difficult problem of “crazy separation, difficult stability” of the progeny of cotton distant hybrids, and after treatment by the method of the present disclosure, the progeny of crosses between G. barbadense and G. hirsutum can be induced to stabilize as soon as possible, and intermediate materials of G. barbadense-G. hirsutum hybrids that conform to the breeding objectives can be systematically selected and bred out from the processed progeny.

In order to realize the above objects, the present disclosure adopts the following technical solution:

A breeding method for rapidly stabilizing cotton distant hybridization progeny includes the following steps:
(1) Crossing an G. barbadense semi-compatible material VSG as the female parent and a G. hirsutum material as the male parent to obtain a F1 generation G. barbadense-G. hirsutum hybrid material;
(2) Treating young bolls of the F1 generation G. barbadense-G. hirsutum hybrid material with colchicine to obtain a F2 generation G. barbadense-G. hirsutum hybrid material; and
(3) Planting all successfully induced bolls, and carrying out 2˜3 generations of continuous selection until the line is stable.

Beneficial effects achieved: the disclosure is based on VSG (G. barbadense semi-compatible material) as the female parent, general G. hirsutum materials as the male parent of the cross to get the F1 generation of G. barbadense-G. hirsutum hybrid materials, and treats the F1 generation of young bolls with 0.03-0.05% of colchicine aqueous solution to induce the F2 generation to separate the stable intermediate materials or new varieties with excellent genes of both G. barbadense and G. hirsutum, so as to produce new cotton germplasm resources.

Further, the G. barbadense semi-compatible material VSG is G. barbadense semi-compatible material VSg: G. barbadense with V7V7 bud yellow gene.

The semi-compatible material (VSG) used in the present disclosure is the G. barbadense type (G. barbadense, 2n=52) material with bud yellow marker gene introduced by the Nanyang Academy of Agricultural Sciences (former Nanyang Institute of Agricultural Sciences) in the early 80's from the United States, and after many years of domestication and planting, the adaptability has been greatly improved.

Further, the G. hirsutum material is an early maturing G. hirsutum variety;
Preferably, the G. hirsutum material is Lumianyan 28 and Jinke 178.

Further, the specific operation of treating with colchicine in the step (2) is as follows: after flowering and boll formation of the plants of the F1 generation of G. barbadense-G. hirsutum hybrid material, selecting the young bolls that are 10˜15 days old after flowering to be processed, and using a 5 ml medical syringe to pierce into middle of the bolls from the base one-third of the young bolls and then injecting 2 ml of aqueous solution of colchicine with mass concentration of 0.03˜0.05%. After the injection treatment, if the cotton bolls develop normally and expand into G. hirsutum boll shape (in marked contrast to the thinness and length of untreated cotton bolls), it indicates that the induction is successful.

Beneficial effects achieved: in the existing technology, the chromosome doubling object is a haploid seedling growth point or adult plant growth point, there are the following defects: first, there is a time limit, and second, the doubling object is a haploid, and the appearance chance of the doubling object is very low. And the method of the present disclosure overcomes the above defects.

After the cotton bolls naturally mature and spit out fluff, the seeds are retained and planted in the following year, and then in accordance with the general systematic breeding methods for 2 to 3 generations, continuous selection is carried out until the line is stabilized into a new line. In this way, the “key” step of inducing G. barbadense-G. hirsutum hybrid progeny to produce haploid is reduced, the induction on the G. barbadense-G. hirsutum hybrid F1 generation of cotton bolls is carried out directly, so that the progeny material is stabilized as soon as possible to achieve the breeding goals.

Further, the specific operation of the step (3) is as follows: after the treated cotton bolls with obvious expansion characteristics naturally mature and spit out fluff, retaining seeds individually, and then planting all the seedlings in the following year, selecting and harvesting G. hirsutum type single plants with early maturity, disease resistance and strong bolling from the progeny group, weighing the weight of a single boll after receiving and drying it, and carrying out an indoor seed test one by one, carding fiber length, ginning lint to calculating the lint percentage, selecting and remaining seed of single plant with boll weight more than 6.0 g, lint percentage greater than 42% and fiber length greater than 30 mm; observing each adult plant row that meets the conditions in the second year, conducting the indoor seed test again after the plants with the same appearance are mixed according to the rows, and breeding the plants that meet the breeding objectives to form a line, and carrying out line identification and variety comparison test.

According to the above technical scheme, compared with the prior art, the disclosure has the following beneficial effects: the main innovation of the disclosure lies in that it changes the previous treatment position and method of colchicine on cotton plant, the previous doubling objects are haploids produced by F1 generation generated by hybrids between semi-compatible materials and G. hirsutum, and the appearance chance of the doubling object is very low. And the disclosure is not limited by whether the haploid can occur in the F1 generation, and directly processes the cotton bolls on the F1 cotton plant, rather than doubling the chromosomes. The purpose of using colchicine is to induce the gene recombination and exchange of F1 generation of G. barbadense-G. hirsutum hybrid to be stable as soon as possible.

### BRIEF DESCRIPTION OF THE DRAWINGS

In order to more clearly illustrate the technical solutions in the embodiments or prior art of the present disclosure, the accompanying drawings to be used in the description of the embodiments or prior art will be briefly introduced below, and it will be obvious that the accompanying drawings in the following description are only embodiments of the present disclosure, and that for the person of ordinary skill in the field, other attachments can be obtained in accordance with the accompanying drawings provided without creative labor.

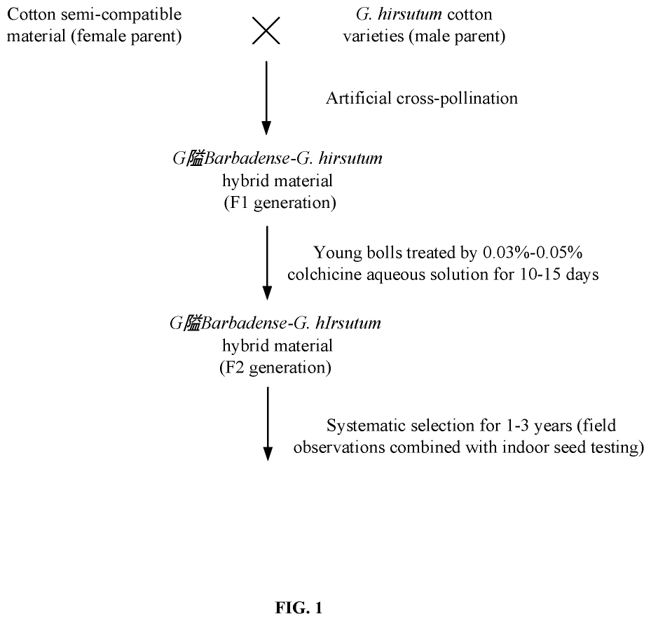

The accompanying drawings of FIG. 1 show a flowchart of the selection and breeding method of the present disclosure.

### DETAILED DESCRIPTION OF THE EMBODIMENTS

The technical solutions in the embodiments of the present disclosure will be clearly and completely described below in conjunction with the accompanying drawings in the embodiments of the present disclosure, and it is clear that the described embodiments are only a part of the embodiments of the present disclosure and not all of the embodiments. Based on the embodiments in the present disclosure, all other embodiments obtained by a person of ordinary skill in the art without making creative labor are within the scope of protection of the present disclosure.

The agents required for the present disclosure are conventional experimental agents procured from commercially available sources; the unmentioned experimental methods are conventional experimental methods and will not be repeated herein.

### Embodiment 1

The method of the present disclosure has been experimentally verified, and the method is feasible.

In 2012, the project team used Lumianyan 28 preferred line, Jinke 178 and other materials as the male parent to cross with VSG material respectively. In 2013, except for dark green leaves, all the other appearance phases of F1 generation plants were female parent type, with vigorous vegetative growth, tall plants, large leaves and deep cracks, large, long and milky white corolla, long oval bolls and thin bolls. The young bolls of 10-15 days old were treated by injecting 2 ml of aqueous solution of colchicine with a concentration of 0.05% per boll, and 5-8 bolls were treated in each combination. After treatment, except for part of the treated cotton bolls dried up and fall off, the appearance of most of the bolls had no change like untreated bolls, and only 1 or 2 bolls in each combination obviously expanded like G. hirsutum bolls. After these bolls naturally matured and spit out fluff, the seeds were retained individually. In 2013, the seeds of each boll were planted into rows, all of which showed the same appearance as G. hirsutum, but there were differences between individuals. In 2014˜2016, single-plant selection of the progeny materials is continued, and in 2017, stable line Wan 116 is selected, and in 2020, stable line Wan 076 is selected, which has the characteristics of high and stable yield, high lint percentage, concentrated bolling and spitting out fluff, and high quality through variety test and identification from 2018 to 2022. Among them, Wan 116 lint cotton was tested by the Cotton Quality Supervision, Inspection and Testing Center of the Ministry of Agriculture, with an average fiber length of 30.3˜31.6 mm in the upper part, which is 2˜3 mm higher than that of Lumianyan 28, with a Micronaire value of about 4.6, which decreased by about 0.4.

In comparison, cotton semi-compatible materials and colchicine are used in previous studies and the disclosure, but the differences in the disclosure are also very obvious: First, the treatment objects are different, previous studies used semi-compatible materials as female parents and low-generation G. barbadense-G. hirsutum hybrid materials as male parents to induce the target haploid, then 0.03-0.05% colchicine was used to treat the target haploid seedlings or adult plants to obtain double G. barbadense-G. hirsutum hybrid stable “homozygous lines” to achieve the breeding goal. Second, the treatment position of plant body are different, the disclosure also uses the semi-compatible materials as the female parents and the G. barbadense-G. hirsutum hybrid low-generation materials as the male parents, but does not first induce haploid and then double the haploid, but directly uses the same dose of colchicine to treat the young on F1 cotton plants, and induces G. barbadense-G. hirsutum hybrid F2 and produced stable G. hirsutum-based phenotypic G. barbadense-G. hirsutum hybrid materials containing G. hirsutum and G. barbadense recombinant genes, so as to achieve the breeding goal.

Each embodiment in this specification is described in a progressive manner, and each embodiment focuses on the differences with other embodiments, and the same and similar parts of each embodiment can be referred to each other.

The above description of the disclosed embodiments enables a person skilled in the art to realize or use the present disclosure. Various modifications to these embodiments will be apparent to those skilled in the art, and the general principles defined herein may be realized in other embodiments without departing from the spirit or scope of the present disclosure. Accordingly, the present disclosure will not be limited to these embodiments shown herein, but will be subject to the broadest scope consistent with the principles and novel features disclosed herein.

What is claimed is:

1. A breeding method for rapidly stabilizing cotton distant hybridization progeny, comprising:
(1) Crossing an G. barbadense semi-compatible material VSG as the female parent and a G. hirsutum material as the male parent to obtain a F1 generation G. barbadense-G. hirsutum hybrid material;
(2) Treating young bolls of the F1 generation G. barbadense-G. hirsutum hybrid material with colchicine to obtain a F2 generation G. barbadense-G. hirsutum hybrid material; and
(3) Planting all successfully induced bolls, and carrying out 2˜3 generations of continuous selection until the line is stable.

2. The breeding method for rapidly stabilizing cotton distant hybridization progeny of claim 1, wherein the G. barbadense semi-compatible material VSG is G. barbadense semi-compatible material VSg: G. barbadense with V7V7 bud yellow gene.

3. The breeding method for rapidly stabilizing cotton distant hybridization progeny of claim 1, wherein the G. hirsutum material is an early maturing G. hirsutum variety;
Preferably, the G. hirsutum material is Lumianyan 28 and Jinke 178.

4. The breeding method for rapidly stabilizing cotton distant hybridization progeny of claim 1, wherein a specific operation of treating with colchicine in the step (2) is as follows: after flowering and boll formation of the plants of the F1 generation of G. barbadense-G. hirsutum hybrid material, selecting young bolls that are 10˜15 days old after flowering to be processed, and using a 5 ml medical syringe to pierce into middle of the bolls from the base one-third of the young bolls and then injecting 2 ml of aqueous solution of colchicine with mass concentration of 0.03˜0.05%.

5. The breeding method for rapidly stabilizing cotton distant hybridization progeny of claim 1, wherein a specific operation of the step (3) is as follows: after the treated cotton bolls with obvious expansion characteristics naturally mature and spit out fluff, retaining seeds individually, and then planting all the seedlings in the following year, selecting and harvesting G. hirsutum type single plants with early maturity, disease resistance and strong bolling from the progeny group, weighing a weight of a single boll after receiving and drying it, and carrying out an indoor seed test one by one, carding fiber length, ginning lint to calculating the lint percentage, selecting and remaining seed of single plant with boll weight more than 6.0 g, lint percentage greater than 42% and fiber length greater than 30 mm;
observing each adult plant row that meets the conditions in the second year, conducting the indoor seed test again after the plants with the same appearance are mixed according to the rows, and breeding the plants that meet the breeding objectives to form a line, and carrying out line identification and variety comparison test.
